(19) United States
(12) Patent Application Publication
(10) Pub. No.: US 2009/0253161 A1
(43) Pub. Date: Oct. 8, 2009

(54) FLUORESCENT PROCHELATORS FOR CELLULAR IRON DETECTION

(75) Inventors: Katherine J. Franz, Durham, NC (US); Lynne M. Hyman, Durham, NC (US)

Correspondence Address: MYERS BIGEL SIBLEY & SAJOVEC, PO BOX 37428, RALEIGH, NC, 27627, US

(73) Assignee: Duke University, Durham, NC (US)

(21) Appl. No.: 12/417,052

(22) Filed: Apr. 2, 2009

Related U.S. Application Data
(60) Provisional application No. 61/042,047, filed on Apr. 3, 2008.

(51) Int. Cl. C12Q 1/02; C07C 241/00

(52) U.S. Cl. 435/29; 564/148

(57) ABSTRACT

Fluorescent probe compound of Formula Ia or Formula Ib:

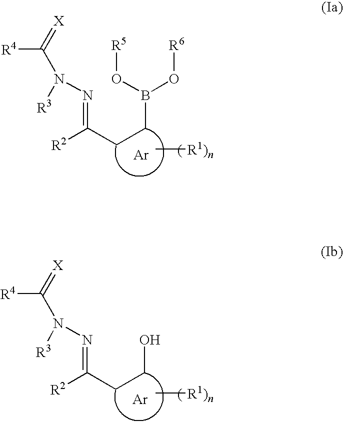

are described, along with methods of using the same to detect iron, copper, and hydrogen peroxide.

### RELATED APPLICATIONS

This application claims the benefit of U.S. Provisional Patent Application Ser. No. 61/042,047, filed Apr. 3, 2009, the disclosure of which is incorporated by reference herein in its entirety.

### FIELD OF THE INVENTION

The present invention concerns fluorescent probe compounds and methods of detecting iron, copper, and hydrogen peroxide therewith.

### BACKGROUND OF THE INVENTION

There are considerable indications for the role of iron in promoting oxidative stress, and yet there remain many questions about the location and speciation of this iron, as well as what conditions lead to its availability for toxic reactions. It also remains to be determined whether iron-promoted oxidative stress is a cause or a consequence of certain diseases. Fluorescent probes that report on the changes of iron status before, during or after oxidative insult to cells have the potential to contribute greatly to this field.

Fluorescent probes offer many advantages for monitoring cellular processes in real time and several have been developed for metal ions such as Ca^{II}, Zn^{II}, Cu^{I}, Hg^{II}. Existing probes for iron include the commercially available calcein and PhenGreen, as well as those in development like CP655 and RDA.¹⁻¹⁰ All of the iron probes work by a fluorescence “turn-off” mechanism resulting from iron-induced fluorescence quenching. In order to measure labile iron in cells by using these probes, a high-affinity chelator is used to compete iron away from the probe and restore its fluorescence. This change upon dequenching provides the concentration of iron that was accessible to the probe chelator.¹¹ A concern with any of these probes is that their mere presence shifts the equilibrium of iron and other metals in the cell by acting as a “vacuum cleaner” that labilizes iron from stores and redistributes it. While calcein is the most widely used probe for iron detection, it is not very selective for iron. Its chelating moiety is similar to EDTA, a broad-spectrum metal chelator. Iron-calcein complexes are also known to be redox active and actually promote Fenton chemistry.¹²

### SUMMARY OF THE INVENTION

Probes that provide either a turn-on or a ratiometric signal without having to add an additional chelator to the cell would be a significant advance in this field, as would reactivity-based probes that could discriminate against the vacuum cleaner effect by triggering only under certain conditions. Our fluorescent prochelator probes provide a novel approach in which reactivity (i.e. oxidative stress) triggers a change in ratiometric fluorescence response that will assay iron in cells and tissues undergoing oxidative stress.

Design of Multifunctional Fluorescent Probes. The general design for multifunctional fluorescent probes that enable visualization of the cellular localization of the pro-chelators as well as sense both H₂O₂ and iron inside cells is shown in schematic form in FIG. 1. These tri-functional fluorescent probes (Flo-B) will be assembled by incorporating fluorescence resonance energy transfer (FRET) donor and acceptor dyes onto the aroylhydrazone pro-chelator core structures. The intact Flo-B probes are designed to have a strong FRET signal, giving emission from the acceptor fluorophore following excitation of the donor. When the probe comes in contact with H₂O₂, the donor fragment attached to the boronate masking group will be released, thereby decreasing FRET and recovering the fluorescence of each individual fluorophore. The change in signal will act as a sensor of cellular H₂O₂. A further change in signal will be provided by iron chelation, which will quench the fluorescence of the fluorophore linked to the ligand but not alter the fluorescence of the released masking group. The ratio of these two fragments will therefore act as a sensor of labile iron. The advantage of having an internal signal against which to ratio the iron-quenched signal provides a significant advantage over existing iron sensors that rely on a secondary chelator to dequench, as described above.

Among other things, the present invention provides a fluorescent probe compound of Formula Ia or Formula Ib:

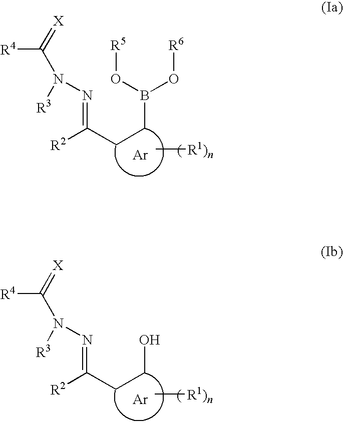

wherein:

R⁴ is A- or A-L-;

X is O or S;

Ar is aryl;

n is an integer from 1 to 4;

each R¹ is independently selected from the group consisting of: alkyl, alkenyl, alkynyl, cycloalkyl, cycloalkylalkyl, cycloalkylalkenyl, cycloalkylalkynyl, heterocyclo, heterocycloalkyl, heterocycloalkenyl, heterocycloalkynyl, aryl, arylalkyl, arylalkenyl, arylalkynyl, heteroaryl, heteroarylalkyl, heteroarylalkenyl, heteroarylalkynyl, alkoxy, halo, mercapto, azido, cyano, formyl, carboxylic acid, hydroxyl, nitro, acyl, aryloxy, alkylthio, amino, alkylamino, arylalkylamino, disubstituted amino, acylamino, acyloxy, ester, amide, sulfoxyl, sulfonyl, sulfonate, sulfonic acid, sulfonamide, urea, alkoxylacylamino, and aminoacyloxy;

R² and R³ are each independently H, alkyl, or hydroxy;

R⁵ and R⁶ are independently selected H, alkyl, or haloalkyl, or together form an alkylene bridge (optionally containing a fused aryl ring), which alkylene bridge may be unsubstituted or substituted from 1 to 4 times with alkyl, halo, a fused aryl ring (such as a fused phenyl), A′-, or A′-L′-,

or each of R⁵ or R⁶ is an independently selected from A′- and A′-L′-;

L and L′ are linking groups; and

A and A′ are first and second fluorophores, which fluorophores are members of a fluorescence resonance energy transfer (FRET) fluorophore pair; or a physiologically-acceptable salt thereof.

In some embodiments of the foregoing, wherein said compound is a compound of Formula Ia, and R⁵ and R⁶ form an alkylene bridge, which alkylene bridge is substituted from 1-4 times with A′-, or A′-L′-.

In some embodiments of the foregoing, wherein said compound is a compound of Formula Ia, and both of R⁵ and R⁶ is independently selected from A′- and A′-L′-.

In some embodiments of the foregoing, wherein said compound is a compound of Formula Ia, and one of R⁵ and R⁶ is independently selected from A′- and A′-L′-.

In some embodiments of the foregoing, wherein said compound is a compound of Formula Ia, and R⁵ and R⁶ are independently selected H, alkyl, or haloalkyl, or together form an alkylene bridge (optionally containing a fused aryl ring), which alkylene bridge may be unsubstituted or substituted from 1 to 4 times with alkyl, halo, a fused aryl ring (such as a fused phenyl).

In some embodiments of the foregoing, said compound is a compound of Formula Ib.

In some embodiments of the foregoing, the compound has the structure of Formula A, B, C, A′, B′ or C′:

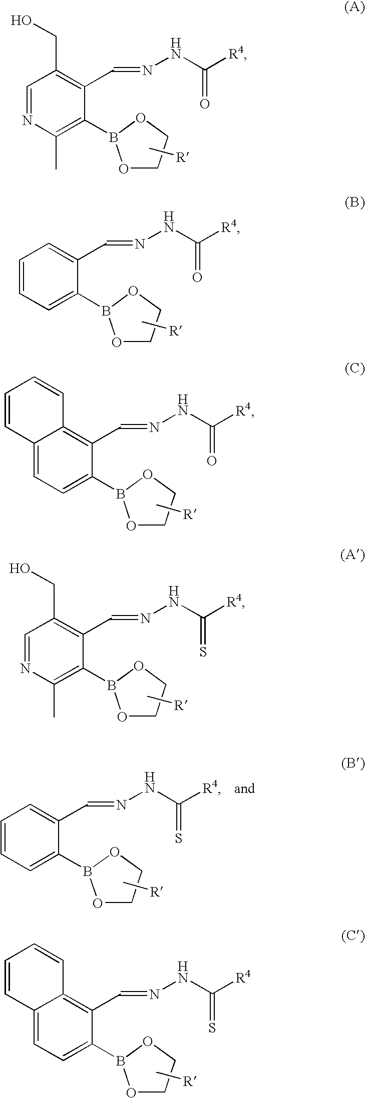

where R⁴ is A- or A-L- as given above, and R′ is A′- or A′-L′- as given above.

In some embodiments of the foregoing, each member of said FRET pair is independently selected from the group consisting of fluorescin dyes, coumarin dyes, xanthine dyes, rhodamine dyes, and cyanine dyes.

In some embodiments of the foregoing, each member of said FRET pair is independently selected from the group consisting of sulfonated cyanine dyes, sulfonated rhodamine dyes, and sulfonated carbocyanine dyes.

A further aspect of the invention is the use of a compound as described above as a fluorescent probe for detecting iron, copper, and/or hydrogen peroxide in a cell or composition.

A further aspect of the invention is a method of detecting the presence or absence of hydrogen peroxide in a cell or composition, comprising: administering a compound of as described above to a cell, or adding a compound of as described above to a composition, under conditions in which said compound is cleaved by hydrogen peroxide in said cell or composition; exciting one member of said FRET pair (A, A′); and detecting emission from the other member of said FRET pair, wherein emission from said other member of said FRET pair is reduced by the cleavage of said compound by hydrogen peroxide in said cell or composition.

A further aspect of the invention is a method of detecting the presence or absence of iron or copper in a cell or composition, comprising: administering a compound of as described above to a cell or composition; exciting said fluorophore A; and detecting the presence or absence of emission from said fluorophore A; wherein emission from fluorophore A is quenched by the presence of iron or copper in said cell or composition.

The foregoing methods can be carried out separately or in combination with one another.

A further aspect of the invention is the compound as described above conjugated to iron or copper.

A further aspect of the invention is a method of detecting the presence or absence of an iron or copper chelating agent in a cell or composition, comprising: administering a compound as described above that is conjugated to iron or copper to a cell or composition, and detecting fluorescence from said compound, where said fluorescence is quenched in the absence of said chelating agent.

A further aspect of the invention is a kit comprising a compound as described above, and/or instructions for carrying out a method as described above, optionally in a common package or container.

The present invention is explained in further detail below.

### BRIEF DESCRIPTION OF THE DRAWINGS

FIG. 1. The generic plan for tri-functional probes Flo-B, which are designed to provide 3 distinct fluorescence responses to report on cellular localization and to detect both H₂O₂ and iron: (a) the intact probe will have strong emission from the acceptor dye (A) upon excitation of the donor (D), (b) H₂O₂ will cleave the donor-containing fragment on the masking group, reducing the FRET signal while maintaining independent signatures of each fluorophore, (c) iron binding to the unmasked chelator will quench the fluorescence of A, while the D fragment will remain fluorescent.

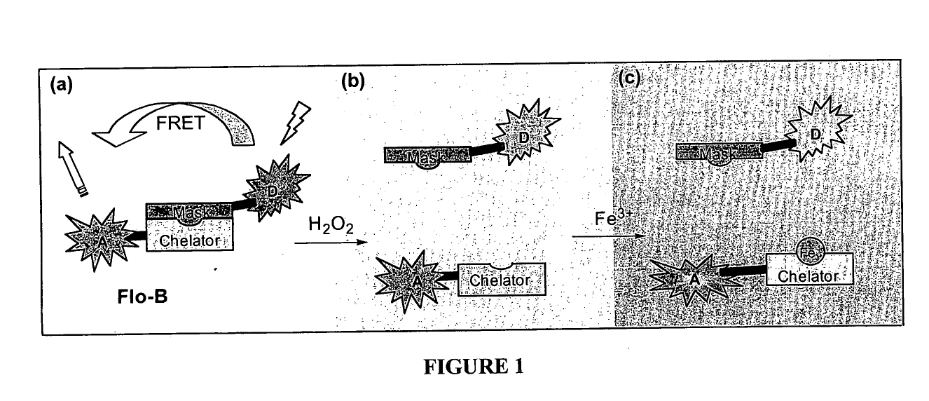

FIG. 2. Target trifunctional probe Flo-B1.

FIG. 3 shows preliminary evidence that Flo-SBH reduces emission in the presence of Fe³⁺.

FIGS. 4A-C show iron binding of Flo-SIH (also referred to as Flo-SBH herein) (FIG. 4A), Flo-BIH (FIG. 4B) and Flo-B (FIG. 4B).

FIG. 5 illustrates the conversion of Flo-B to Flo-SIH by H₂O₂.

FIGS. 6A-B illustrate iron induced fluorescent quenching of Flo-SIH, but not Flo-B or Flo-BIH.

FIGS. 7A-B illustrate the metal-dependent quenching of Flo-SIH.

### DETAILED DESCRIPTION OF THE INVENTION

“Alkyl” as used herein alone or as part of another group, refers to a straight or branched chain hydrocarbon containing from 1 to 10 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, and the like. “Lower alkyl” as used herein, is a subset of alkyl, in some embodiments preferred, and refers to a straight or branched chain hydrocarbon group containing from 1 to 4 carbon atoms. Representative examples of lower alkyl include, but are not limited to, methyl, ethyl, n-propyl, iso-propyl, n-butyl, iso-butyl, tert-butyl, and the like. The term “akyl” or “loweralkyl” is intended to include both substituted and unsubstituted alkyl or loweralkyl unless otherwise indicated and these groups may be substituted with groups selected from halo (e.g., haloalkyl), alkyl, haloalkyl, alkenyl, alkynyl, cycloalkyl, cycloalkylalkyl, aryl, arylalkyl, heterocyclo, heterocycloalkyl, hydroxyl, alkoxy (thereby creating a polyalkoxy such as polyethylene glycol), alkenyloxy, alkynyloxy, haloalkoxy, cycloalkoxy, cycloalkylalkyloxy, aryloxy, arylalkyloxy, heterocyclooxy, heterocyclolalkyloxy, mercapto, alkyl-S(O)ₘ, haloalkyl-S(O)ₘ, alkenyl-S(O)ₘ, alkynyl-S(O)ₘ, cycloalkyl-S(O)ₘ, cycloalkylalkyl-S(O)ₘ, aryl-S(O)ₘ, arylalkyl-S(O)ₘ, heterocyclo-S(O)ₘ, heterocycloalkyl-S(O)ₘ, amino, carboxy, alkylamino, alkenylamino, alkynylamino, haloalkylamino, cycloalkylamino, cycloalkylalkylamino, arylamino, arylalkylamino, heterocycloamino, heterocycloalkylamino, disubstituted-amino, acylamino, acyloxy, ester, amide, sulfonamide, urea, alkoxyacylamino, aminoacyloxy, nitro or cyano where m=0, 1, 2 or 3.

“Alkenyl” as used herein alone or as part of another group, refers to a straight or branched chain hydrocarbon containing from 1 to 10 carbon atoms (or in loweralkenyl 1 to 4 carbon atoms) which include 1 to 4 double bonds in the normal chain. Representative examples of alkenyl include, but are not limited to, vinyl, 2-propenyl, 3-butenyl, 2-butenyl, 4-pentenyl, 3-pentenyl, 2-hexenyl, 3-hexenyl, 2,4-heptadiene, and the like. The term “alkenyl” or “loweralkenyl” is intended to include both substituted and unsubstituted alkenyl or loweralkenyl unless otherwise indicated and these groups may be substituted with groups as described in connection with alkyl and loweralkyl above.

“Alkynyl” as used herein alone or as part of another group, refers to a straight or branched chain hydrocarbon containing from 1 to 10 carbon atoms (or in loweralkynyl 1 to 4 carbon atoms) which include 1 triple bond in the normal chain. Representative examples of alkynyl include, but are not limited to, 2-propynyl, 3-butynyl, 2-butynyl, 4-pentynyl, 3-pentynyl, and the like. The term “alkynyl” or “loweralkynyl” is intended to include both substituted and unsubstituted alkynyl or loweralknynyl unless otherwise indicated and these groups may be substituted with the same groups as set forth in connection with alkyl and loweralkyl above.

“Cycloalkyl” as used herein alone or as part of another group, refers to a saturated or partially unsaturated cyclic hydrocarbon group containing from 3, 4 or 5 to 6, 7 or 8 carbons (which carbons may be replaced in a heterocyclic group as discussed below). Representative examples of cycloalkyl include, cyclopropyl, cyclobutyl, cyclopentyl, cyclohexyl, cycloheptyl, and cyclooctyl. These rings may be optionally substituted with additional substituents as described herein such as halo or loweralkyl. The term “cycloalkyl” is generic and intended to include heterocyclic groups as discussed below unless specified otherwise.

“Heterocyclo” as used herein alone or as part of another group, refers to an aliphatic (e.g., fully or partially saturated heterocyclo) or aromatic (e.g., heteroaryl) monocyclic- or a bicyclic-ring system. Monocyclic ring systems are exemplified by any 5 or 6 membered ring containing 1, 2, 3, or 4 heteroatoms independently selected from oxygen, nitrogen and sulfur. The 5 membered ring has from 0-2 double bonds and the 6 membered ring has from 0-3 double bonds. Representative examples of monocyclic ring systems include, but are not limited to, azetidine, azepine, aziridine, diazepine, 1,3-dioxolane, dioxane, dithiane, furan, imidazole, imidazoline, imidazolidine, isothiazole, isothiazoline, isothiazolidine, isoxazole, isoxazoline, isoxazolidine, morpholine, oxadiazole, oxadiazoline, oxadiazolidine, oxazole, oxazoline, oxazolidine, piperazine, piperidine, pyran, pyrazine, pyrazole, pyrazoline, pyrazolidine, pyridine, pyrimidine, pyridazine, pyrrole, pyrroline, pyrrolidine, tetrahydrofuran, tetrahydrothiophene, tetrazine, tetrazole, thiadiazole, thiadiazoline, thiadiazolidine, thiazole, thiazoline, thiazolidine, thiophene, thiomorpholine, thiomorpholine sulfone, thiopyran, triazine, triazole, trithiane, and the like. Bicyclic ring systems are exemplified by any of the above monocyclic ring systems fused to an aryl group as defined herein, a cycloalkyl group as defined herein, or another monocyclic ring system as defined herein. Representative examples of bicyclic ring systems include but are not limited to, for example, benzimidazole, benzothiazole, benzothiadiazole, benzothiophene, benzoxadiazole, benzoxazole, benzofuran, benzopyran, benzothiopyran, benzodioxine, 1,3-benzodioxole, cinnoline, indazole, indole, indoline, indolizine, naphthyridine, isobenzofuran, isobenzothiophene, isoindole, isoindoline, isoquinoline, phthalazine, purine, pyranopyridine, quinoline, quinolizine, quinoxaline, quinazoline, tetrahydroisoquinoline, tetrahydroquinoline, thiopyranopyridine, and the like. These rings include quaternized derivatives thereof and may be optionally substituted with groups selected from halo, alkyl, haloalkyl, alkenyl, alkynyl, cycloalkyl, cycloalkylalkyl, aryl, arylalkyl, heterocyclo, heterocycloalkyl, hydroxyl, alkoxy, alkenyloxy, alkynyloxy, haloalkoxy, cycloalkoxy, cycloalkylalkyloxy, aryloxy, arylalkyloxy, heterocyclooxy, heterocyclolalkyloxy, mercapto, alkyl-S(O)ₘ, haloalkyl-S(O)ₘ, alkenyl-S(O)ₘ, alkynyl-S(O)ₘ, cycloalkyl-S(O)ₘ, cycloalkylalkyl-S(O)ₘ, aryl-S(O)ₘ, arylalkyl-S(O)ₘ, heterocyclo-S(O)ₘ, heterocycloalkyl-S(O)ₘ, amino, alkylamino, alkenylamino, alkynylamino, haloalkylamino, cycloalkylamino, cycloalkylalkylamino, arylamino, arylalkylamino, heterocycloamino, heterocycloalkylamino, disubstituted-amino, acylamino, acyloxy, ester, amide, sulfonamide, urea, alkoxyacylamino, aminoacyloxy, nitro or cyano where m =0, 1, 2 or 3.

“Aryl” as used herein alone or as part of another group, refers to a monocyclic carbocyclic ring system or a bicyclic carbocyclic fused ring system having one or more aromatic rings. Representative examples of aryl include, azulenyl, indanyl, indenyl, naphthyl, phenyl, tetrahydronaphthyl, and the like. In some embodiments aryl contains a “hetero” atom and is also a “heterocyclo” group as described above. The term “aryl” is intended to include both substituted and unsubstituted aryl unless otherwise indicated and these groups may be substituted with the same groups as set forth in connection with alkyl and loweralkyl above. More specifically, “aryl” groups as used herein may be substituted 1, 2, 3, or 4 or more times with with independently selected halo (e.g., haloaryl), alkyl, haloalkyl, alkenyl, alkynyl, cycloalkyl, cycloalkylalkyl, aryl, arylalkyl, heterocyclo, heterocycloalkyl, hydroxyl, alkoxy (thereby creating a polyalkoxy such as polyethylene glycol), alkenyloxy, alkynyloxy, haloalkoxy, cycloalkoxy, cycloalkylalkyloxy, aryloxy, arylalkyloxy, heterocyclooxy, heterocyclolalkyloxy, mercapto, alkyl-S(O)ₘ, haloalkyl-S(O)ₘ, alkenyl-S(O)ₘ, alkynyl-S(O)ₘ, cycloalkyl-S(O)ₘ, cycloalkylalkyl-S(O)ₘ, aryl-S(O)ₘ, arylalkyl-S(O)ₘ, heterocyclo-S(O)ₘ, heterocycloalkyl-S(O)ₘ, amino, carboxy, alkylamino, alkenylamino, alkynylamino, haloalkylamino, cycloalkylamino, cycloalkylalkylamino, arylamino, arylalkylamino, heterocycloamino, heterocycloalkylamino, disubstituted-amino, acylamino, acyloxy, ester, amide, sulfonamide, urea, alkoxyacylamino, aminoacyloxy, nitro or cyano where m=0, 1, 2 or 3.

“Arylalkyl” as used herein alone or as part of another group, refers to an aryl group, as defined herein, appended to the parent molecular moiety through an alkyl group, as defined herein. Representative examples of arylalkyl include, but are not limited to, benzyl, 2-phenylethyl, 3-phenylpropyl, 2-naphth-2-ylethyl, and the like.

“Heteroaryl” as used herein is as described in connection with heterocyclo above.

“Alkoxy” as used herein alone or as part of another group, refers to an alkyl or loweralkyl group, as defined herein (and thus including substituted versions such as polyalkoxy), appended to the parent molecular moiety through an oxy group, —O—. Representative examples of alkoxy include, but are not limited to, methoxy, ethoxy, propoxy, 2-propoxy, butoxy, tert-butoxy, pentyloxy, hexyloxy and the like.

“Halo” as used herein refers to any suitable halogen, including —F, —Cl, —Br, and —I.

“Mercapto” as used herein refers to an —SH group.

“Azido” as used herein refers to an —N₃ group.

“Cyano” as used herein refers to a —CN group.

“Formyl” as used herein refers to a —C(O)H group.

“Carboxylic acid” as used herein refers to a —C(O)OH group.

“Hydroxyl” as used herein refers to an —OH group.

“Nitro” as used herein refers to an —NO₂ group.

“Acyl” as used herein alone or as part of another group refers to a —C(O)R radical, where R is any suitable substituent such as aryl, alkyl, alkenyl, alkynyl, cycloalkyl or other suitable substituent as described herein.

“Alkylthio” as used herein alone or as part of another group, refers to an alkyl group, as defined herein, appended to the parent molecular moiety through a thio moiety, as defined herein. Representative examples of alkylthio include, but are not limited, methylthio, ethylthio, tert-butylthio, hexylthio, and the like.

“Amino” as used herein means the radical —NH2.

“Alkylamino” as used herein alone or as part of another group means the radical —NHR, where R is an alkyl group.

“Arylalkylamino” as used herein alone or as part of another group means the radical —NHR, where R is an arylalkyl group.

“Disubstituted-amino” as used herein alone or as part of another group means the radical —NRₐR_{b}, where Rₐ and R_{b} are independently selected from the groups alkyl, haloalkyl, alkenyl, alkynyl, cycloalkyl, cycloalkylalkyl, aryl, arylalkyl, heterocyclo, heterocycloalkyl.

“Acylamino” as used herein alone or as part of another group means the radical —NRₐR_{b}, where Rₐ is an acyl group as defined herein and R_{b} is selected from the groups hydrogen, alkyl, haloalkyl, alkenyl, alkynyl, cycloalkyl, cycloalkylalkyl, aryl, arylalkyl, heterocyclo, heterocycloalkyl.

“Acyloxy” as used herein alone or as part of another group means the radical —OR, where R is an acyl group as defined herein.

“Ester” as used herein alone or as part of another group refers to a —C(O)OR radical, where R is any suitable substituent such as alkyl, cycloalkyl, alkenyl, alkynyl or aryl.

“Amide” as used herein alone or as part of another group refers to a —C(O)NRₐR_{b} radical, where Rₐ and R_{b} are any suitable substituent such as alkyl, cycloalkyl, alkenyl, alkynyl or aryl.

“Sulfoxyl” as used herein refers to a compound of the formula —S(O)R, where R is any suitable substituent such as alkyl, cycloalkyl, alkenyl, alkynyl or aryl.

“Sulfonyl” as used herein refers to a compound of the formula —S(O)(O)R, where R is any suitable substituent such as alkyl, cycloalkyl, alkenyl, alkynyl or aryl.

“Sulfonate” as used herein refers to a compounnd of the formula —S(O)(O)OR, where R is any suitable substituent such as alkyl, cycloalkyl, alkenyl, alkynyl or aryl.

“Sulfonic acid” as used herein refers to a compound of the formula —S(O)(O)OH.

“Sulfonamide” as used herein alone or as part of another group refers to a —S(O)2NRₐR_{b} radical, where Rₐ and R_{b} are any suitable substituent such as H, alkyl, cycloalkyl, alkenyl, alkynyl or aryl.

“Urea” as used herein alone or as part of another group refers to an —N(Rc)C(O)NRₐR_{b} radical, where Rₐ, R_{b} and R_{c} are any suitable substituent such as H, alkyl, cycloalkyl, alkenyl, alkynyl or aryl.

“Alkoxyacylamino” as used herein alone or as part of another group refers to an —N(Rₐ)C(O)OR_{b} radical, where Rₐ, R_{b} are any suitable substituent such as H, alkyl, cycloalkyl, alkenyl, alkynyl or aryl.

“Aminoacyloxy” as used herein alone or as part of another group refers to an —OC(O)NRₐR_{b} radical, where Rₐ and R_{b} are any suitable substituent such as H, alkyl, cycloalkyl, alkenyl, alkynyl or aryl.

“Linking group” as used herein may be an aromatic or aliphatic group (including both saturated and unsaturated aliphatic groups) (which may be substituted or unsubstituted and may optionally contain heteroatoms such as N, O, or S) that are utilized to couple a fluorophore to the core structure or parent molecule. (e.g., Formula Ia, Ib). Examples include but are not limited to aryl (such as p-phenylene), alkyl, heteroaryl, heteroalkyl (e.g., oligoethylene glycol), peptide, and, polysaccharide linkers, etc. Some fluorescent dyes are commercially available with corresponding linking groups or conjugatable groups. Additional examples of linking groups include but are not limited to C1-C20 alkynyl-amine, alkynol, alkenamine? alkylamine, keto, and thiol (see, e.g., U.S. Pat. No. 6,855,503)

Fluorescence resonance energy transfer (FRET) is the nonradiative transfer of electronic excitation energy from a donor to an acceptor molecule by a weak dipole-dipole coupling mechanism. When the emission of the donor chromophore overlaps the absorption of the acceptor chromophore, energy can flow from the excited donor to the acceptor, which typically emits a longer wavelength photon than that of the donor. The energy-transfer efficiency (E) decreases inversely proportional to R⁶, the distance (R) between the donor and the acceptors, as E=1/(1+(R/Rₒ)⁶) (the Forster radius, Rₒ, is the distance corresponding to 50% energy transfer and depends on the photophysical properties of the dyes and their relative orientations). Thus, FRET is very sensitive to the distance (R) between fluorescent donors and fluorescent acceptors. Indeed, FRET allows distance measurements to be made on a nanometer scale, typically over a range of from about 2 to about 8 nanometers. Thus, FRET is well suited to the study of biological interactions of macromolecules. See, e.g., U.S. Pat. No. 7,238,792.

“FRET pair” or “FRET fluorophores pair” as used herein refers to a pair of fluorophores (also referred to as “dyes” or “chromophores”) that can engage in fluorescence resonance energy transfer (FRET). In the pair, one fluorophore is typically referred to as the “donor” and the other fluorophore is typically referred to as the “acceptor.” Numerous different fluorophores that can be used in FRET pairs are known. See, e.g., U.S. Pat. Nos. 7,511,811; 7,279,317; 7,273,700; 7,238,792; 7,192,710; 7,067,324; and 6,982,146. In general, compounds suitable for fluorophores include but are not limited to fluorescin dyes, coumarin dyes, xanthine dyes, rhodamine dyes, and cyanine dyes (these terms including derivatives thereof such as sulfonated cyanine dyes, sulfonated rhodamine dyes, sulfonated carbocyanine dyes, etc.). Other suitable dyes include but are not limited to phthalocyanine dyes, phycobiliprotein dyes, pyrene dyes and sulfonated pyrene dyes, squaraine dyes and Lucifer yellow. See, e.g., U.S. Pat. No. 7,511,811. Illustrative donors are BODIPY FL (trade names; products of Molecular Probes Inc., USA), BODIPY 493/503 (trade names; products of Molecular Probes Inc., USA), 5-FAM, Tetramethylrhodamine, 6-TAMRA, Fluorescein and derivatives thereof [for example, flurorescein isothiocyanate (FITC) and its derivatives]; Alexa 488, Alexa 532, cy3, cy5, EDANS (5-(2′-aminoethyl)amino-l-naphtalene sulfonic acid), rhodamine 6G (R6G) and its derivatives [for example, tetramethylrhodamine isothiocyanate (TMRITC)], BODIPY FL/C3 (trade names; products of Molecular Probes Inc., USA), BODIPY FL/C6 (trade names; products of Molecular Probes Inc., USA), BODIPY 5-FAM (trade names; products of Molecular Probes Inc., USA), BODIPY TMR (trade names; products of Molecular Probes Inc., USA) or its derivatives [for example, BODIPY TR (trade names; products of Molecular Probes Inc., USA)], BODIPY R6G (trade names; products of Molecular Probes Inc., USA), BODIPY 564 (trade names; products of Molecular Probes Inc., USA), BODIPY 581/591 (trade names; products of Molecular Probes Inc., USA), Dabcyl (4,4 -Dimethylamino-azobenzene-4′ -carboxylic acid). See, e.g., U.S. Pat. Nos. 7,273,700 and 7,495,069. Acceptor fluorophores for use in the present invention can be any that are capable of serving as an acceptor dye in a pair with a donor, i.e., that are capable of receiving energy transferred from the donor dye. The type of the acceptor dye depends on the type of the donor dye to constitute the pair. For example, X-rhodamine and BODIPY 581/591 (trade name; a product of Molecular Probes Inc., USA)-can be used as the acceptor dye when the donor dye is any of the BODIPY FL series dyes (trade name; a product of Molecular Probes Inc., USA), BODIPY FL-series dyes (trade names; products of Molecular Probes Inc., USA), BODIPY 493/503 (trade name; a product of Molecular Probes Inc., USA), 5-FAM, BODIPY 5-FAM (trade name; a product of Molecular Probes Inc., USA), Tetramethylrhodanine, and 6-TAMRA. Coumarin and derivatives thereof such as DCCH can be used when the donor is fluorescin or a derivative thereof. However, numerous acceptor dyes are known and acceptors for use in the present invention are not limited to these examples. See, e.g., U.S. Pat. No. 7,273,700; see also J. Berlier et al., Quantitative Comparison of Long-wavelength Alexa Fluor Dyes to Cy Dyes: Fluorescence of the Dyes and Their Bioconjugates, J Histochem. & Cytochem. 51, 1699-1712 (2003); N. Panchuk-Voloshina et al., Alexa Dyes, a Series of New Fluorescent Dyes that Yield Exceptionally Bright, Photostable Conjugates, J. Histochem & Cytochem 47, 1179-1188 (1999). Additional examples of suitable fluorescent dyes, including linking groups or conjugatable groups that can be used therewith, include but are not limited to those set forth in U.S. Pat. Nos. 6,977,305; 6,974,873; 6,716,979; and 6,399,392 (all to Molecular Probes); and 6,855,503 (to Amersham).

### 1. Compounds

As noted above, the present invention provides fluorescent probe compound of Formula Ia or Formula Ib:

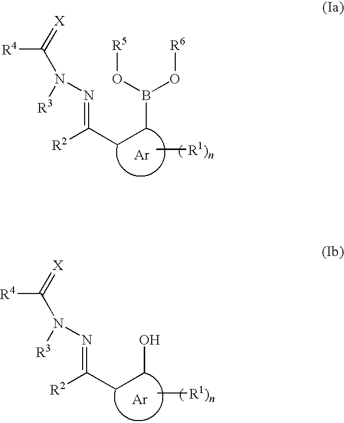

wherein:

Ar is aryl, non-limiting examples of which include monocyclic or fused ring aryl, optionally containing from 1 to 3 hetero atoms selected from N, O, and S (e.g., phenyl, pyridyl, naphthyl)(or stated otherwise, “Ar” represents an aromatic ring system such as benzene, pyridine, naphthalene, etc., which may be substituted or unsubstituted as described hereinabove and below);

n are each 0, 1, 2, 3, or 4 (or in some embodiments not more than 2 or 3 where fewer positions are available for substitution; it being understood that in the case of formula Ic groups R¹ may be substituted on either ring of the naphthyl group);

each R¹ is independently selected from the group consisting of: alkyl, alkenyl, alkynyl, cycloalkyl, cycloalkylalkyl, cycloalkylalkenyl, cycloalkylalkynyl, heterocyclo, heterocycloalkyl, heterocycloalkenyl, heterocycloalkynyl, aryl, arylalkyl, arylalkenyl, arylalkynyl, heteroaryl, heteroarylalkyl, heteroarylalkenyl, heteroarylalkynyl, alkoxy, halo, mercapto, azido, cyano, formyl, carboxylic acid, hydroxyl, nitro, acyl, aryloxy, alkylthio, amino, alkylamino, arylalkylamino, disubstituted amino, acylamino, acyloxy, ester, amide, sulfoxyl, sulfonyl, sulfonate, sulfonic acid, sulfonamide, urea, alkoxylacylamino, and aminoacyloxy (it being understood that R's may be on either ring in the fused ring system shown in Formula Ic);

R² and R³ are each independently H, alkyl, or hydroxy;

R⁵ and R⁶ are independently selected H, alkyl, haloalkyl, or together form an alkylene bridge (e.g., a C2 to C4, C6 or C8 alkylene bridge (e.g., —(CH₂)ₙ—where n is 2 to 4, 6 or 8), which may be unsubstituted or substituted from 1 to 4 times with alkyl, halo, or a fused aryl ring such as a fused phenyl), A′-, or A′-L′-,

or each of R⁵ or R⁶ is an independently selected from A′- and A′-L′-;

L and L′ are linking groups; and

A and A′ are first and second fluorophores, which fluorophores are members of a fluorescence resonance energy transfer (FRET) fluorophore pair.

or a physiologically-acceptable salts-thereof.

In some embodiments R⁵ and R⁶ together form:

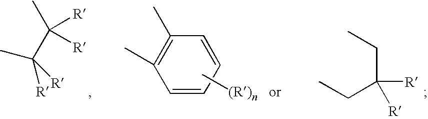

where n is 0, 1, 2, 3 or 4 and each R′ is independently selected from alkyl, halo, A′- or A′-L′-.

Particular examples of compounds of the present invention include, but are not limited to:

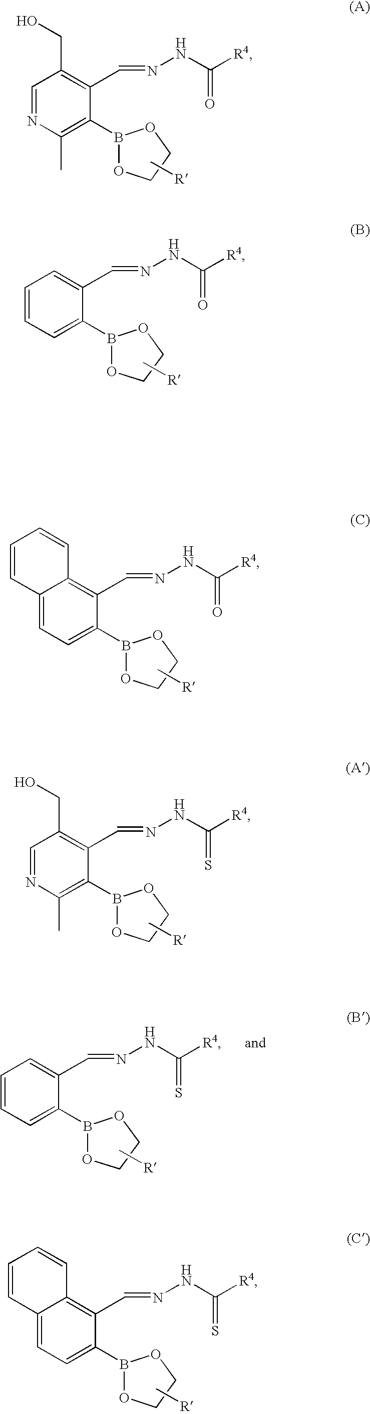

where R⁴ is A- or A-L- as given above, and R′ is A′- or A′-L′-.

Compounds of the present invention can be made by the methods described herein, or variations thereof that will be apparent to those skilled in the art based upon those arts and the present disclosure. See, e.g., Franz and Charkoudian, PCT Patent Application WO 2008/020920; U.S. Pat. Nos. 6,977,305; 6,974,873; 6,716,979; 6,399,392; and 6,855,503.

2. Methods

Cells from which emission may be detected in the present invention include plant, animal, and microbial cells. Cells may be individual cells in vitro or in a cell culture, or cells residing in a tissue, which tissue is in vivo or in vitro in a tissue culture.

Plant cells may be from or of any suitable plant, including angiosperms and gymnosperms, and including monocots and dicots. Exanples include but are not limited to wheat, soy, corn, potato, tomato, orange, lemon, pine, oak, etc.

Animal cells may be from or of any animal, including but not limited to avian, reptile, amphibian, and mammalian species, such as mouse, rat, cat, dog, horse, cow, sheep, rabbit, and primate such as human.

Microbial cells may be from any microorganism, including yeast, fungi, protozoa, and bacteria, including gram negative and gram positive bacteria.

Compositions from which emission may be detected may be any mixtures or materials, typically liquid and more typically aqueous, suspected of containing iron, copper, and/or hydrogen peroxide.

Compounds of the invention may be contacted or administered to cells of compositions as described herein by any suitable technique, such as simply combining with the mixture, adding to a cell or tissue culture, injection into a subject, etc., and fluorescence of FRET measured therefrom in accordance with known techniques or variations thereof which will be apparent to those skilled in the art.

Kits useful for carrying out the present invention may comprise one or more compounds as described herein, and/or instructions (e.g., printed instructions) for carrying out a method as described herein, optionally packaged together in a common package or container.

### Example 1

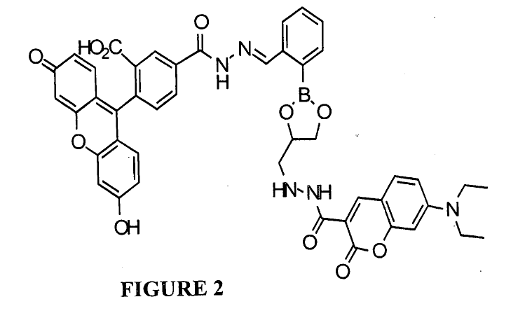

Development of the multifunctional sensors relies on choosing appropriate FRET pairs that flank an iron-binding site and a masking group. Our first target probe, Flo-B1 shown in FIG. 2, contains coumarin as an energy donor appended to the boronic ester of the BSBH core, to which fluorescein is appended as the energy acceptor. The coumarin-fluorescein pair provides efficient FRET,¹³ with excitation and emission wavelengths for coumarin being 420 nm and 468 nm, respectively, while those for fluorescein are 495 nm and 515 nm. The flexibility in the synthetic plan allows us to exchange these fluorophores with more photostable (but more costly) dyes, such as the Alexafluors (Molecular Probes), as we move from in vitro experiments to more demanding cell culture microscopy experiments where photobleaching will be an important consideration.

### Synthesis of Flo-B Components

Before assembling the intact trifunctional Flo-B1, each half will be synthesized independently to allow us to test the quenching efficiency of iron binding to the fluorescent chelator and to verify the stability of the fluorophore-conjugated boronate ester. The fluorescently labeled chelator Flo-SBH (also referred to as Flo-SIH herein) or pro-chelator Flo-BASBH are prepared by reacting salicylaldehyde or (2-formylphenyl)boronic acid with a hydrazine derivative of fluorescein, as shown in Scheme 1. An additional control compound, Flo-BIH, will also be prepared that contains neither phenol nor boronic acid. The fluorescein-hydrazine itself is efficiently prepared by reacting fluorescein succinimidyl ester with hydrazine. We have recently verified the success of this synthetic route by isolating Flo-SBH, as confirmed by NMR and mass spectrometry.

The synthesis of coumarin-appended BSBH (BSBH-Cmn) is outlined in Scheme 2. The first step is a Schiff base condensation between 7-diethylaminocoumarin-3-carboxylic acid hydrazide (DCCH) and DL-glyceraldehyde. DCCH is synthesized following known procedures.¹⁴ The resulting hydrazone will be reduced with NaBH₃CN to give the coumarin-appended diol shown in the scheme, which will be coupled to BASBH to form the boronic ester by refluxing stoichiometric amounts of each in benzene.¹⁵

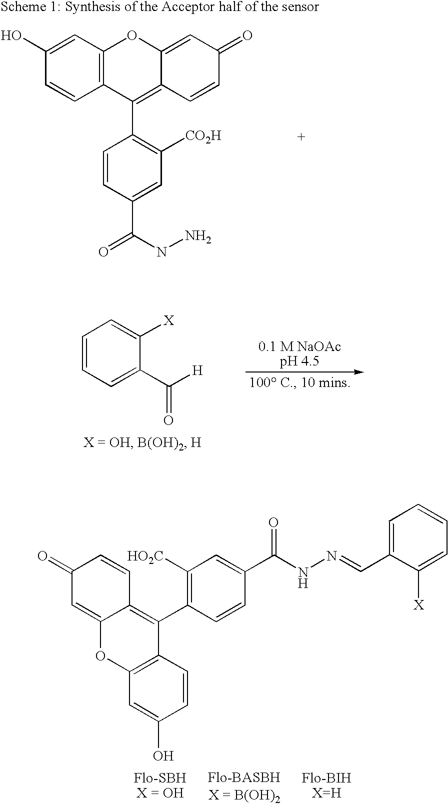

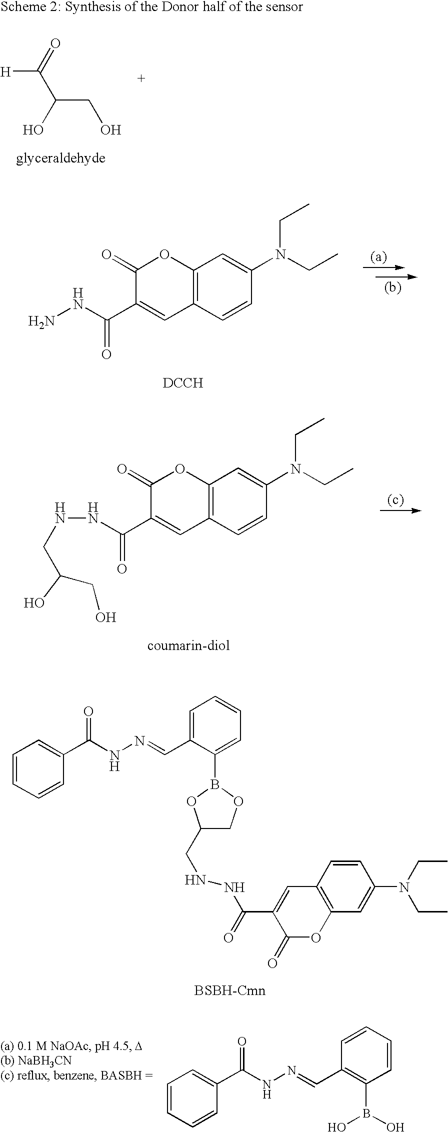

### Flo-SBH as an Iron-Responsive Probe

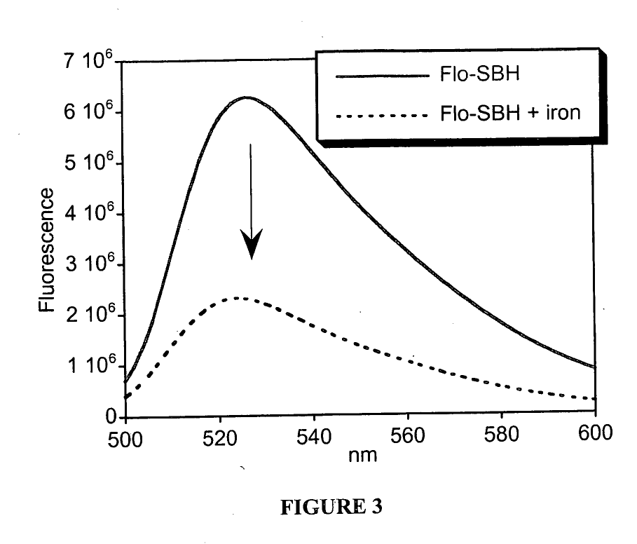

Preliminary evidence that Flo-SBH reduces emission in the presence of Fe³⁺ is shown in FIG. 3. To accurately determine the mole-fraction of iron needed to maximize quenching, we will titrate solutions of Flo-SBH with solutions of Fe³⁺ until maximum quenching is observed. Similar experiments with Ca²⁺, Mg²⁺, Cu²⁺, Cu⁺, Co²⁺, Ni²⁺, Zn²⁺, Mn²⁺, and Fe²⁺ will determine the specificity of the quenching response, while competition experiments that contain non-quenching metal ions in the presence of Fe³⁺ will further characterize the probe's response. This same panel of experiments will then be performed on Flo-BSBH (the boronate-masked version) and Flo-BIH (a non-chelating control) to verify that the iron-induced quenching is specific for the unmasked chelator.

### Example 2

### Synthesis of Flo-B Iron Probes

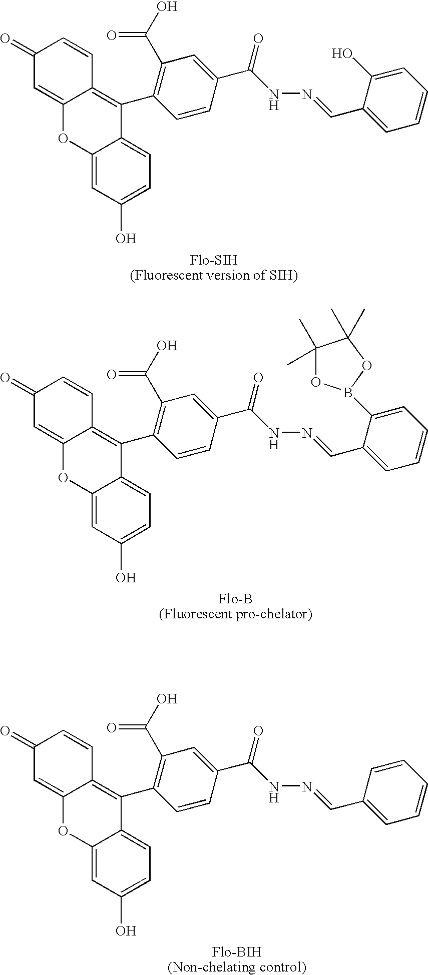

Step 1:

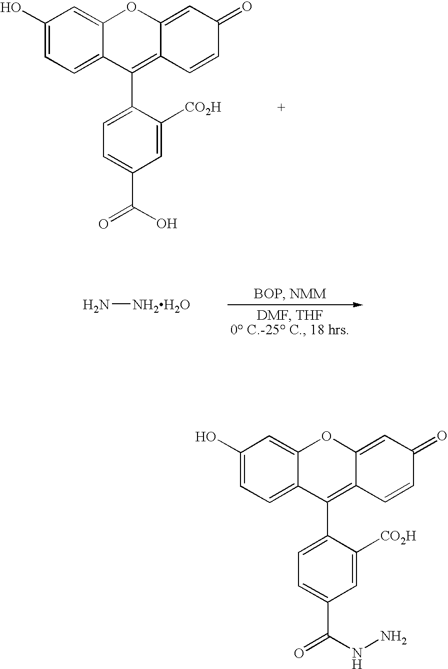

Step 2:

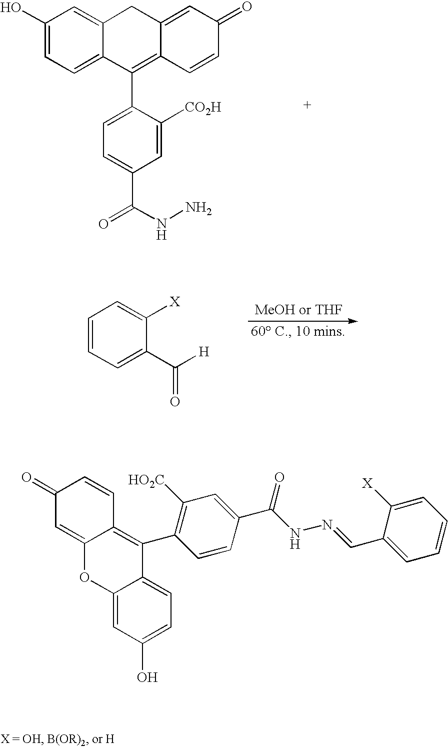

### Iron Binding

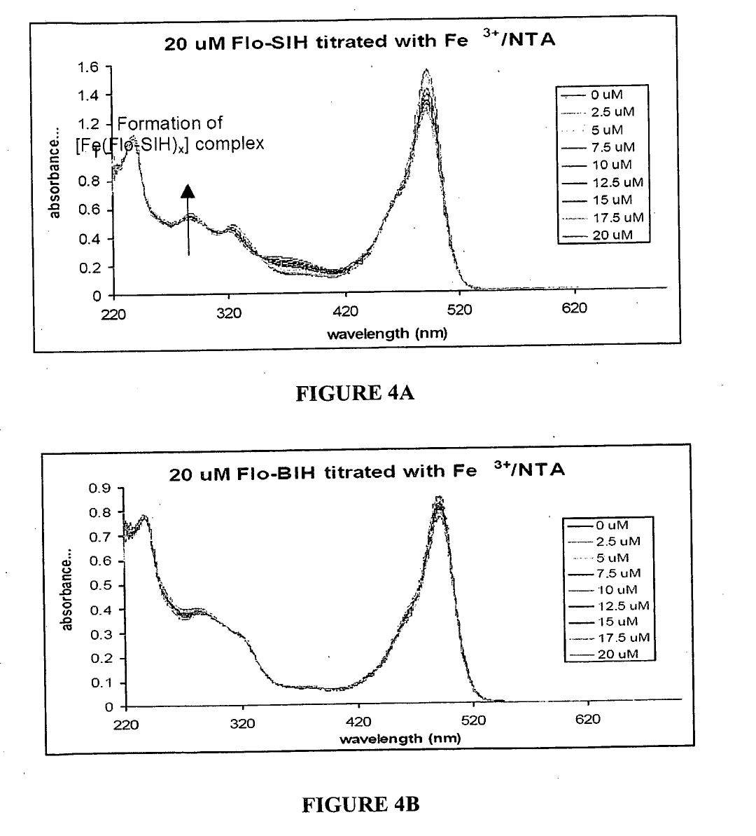

Solutions of 20 uM Flo-SIH (also referred to as Flo-SBH herein), Flo-BIH and Flo-B in 10 mM HEPES/100 mM NaCi buffer at pH 7.00 were titrated with [Fe³⁺(NTA)]. The UV-vis spectra show that Flo-SIH readily extracts Fe³⁺ from Fe(NTA) to form an Fe(Flo-SIH) complex (FIG. 4A), but neither the nonchelating control Flo-BIH nor the pro-chelator Flo-B (FIGS. 4B-C) forms a complex with Fe³⁺. These data are consistent with the design of the probe to bind iron only in the unmasked form.

### Conversion of Flo-B to Flo-SIH by H₂O₂

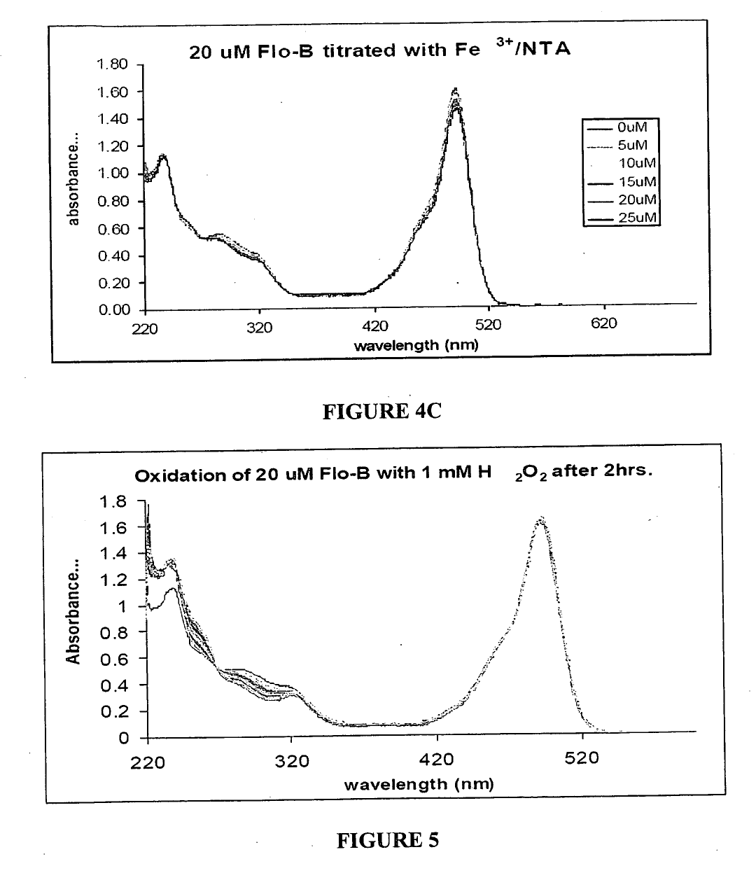

1 mM H₂O₂ was added to a 20 uM solution of Flo-B and monitored for 2 hours with UV-Vis spectrometry. As shown in FIG. 5, conversion was seen corresponding to oxidation of the boronic ester masking group, revealing the phenol needed for iron binding. Mass spectroscopy of the product confirms that Flo-B was converted to Flo-SIH.

### Iron Induced Fluorescent Quenching

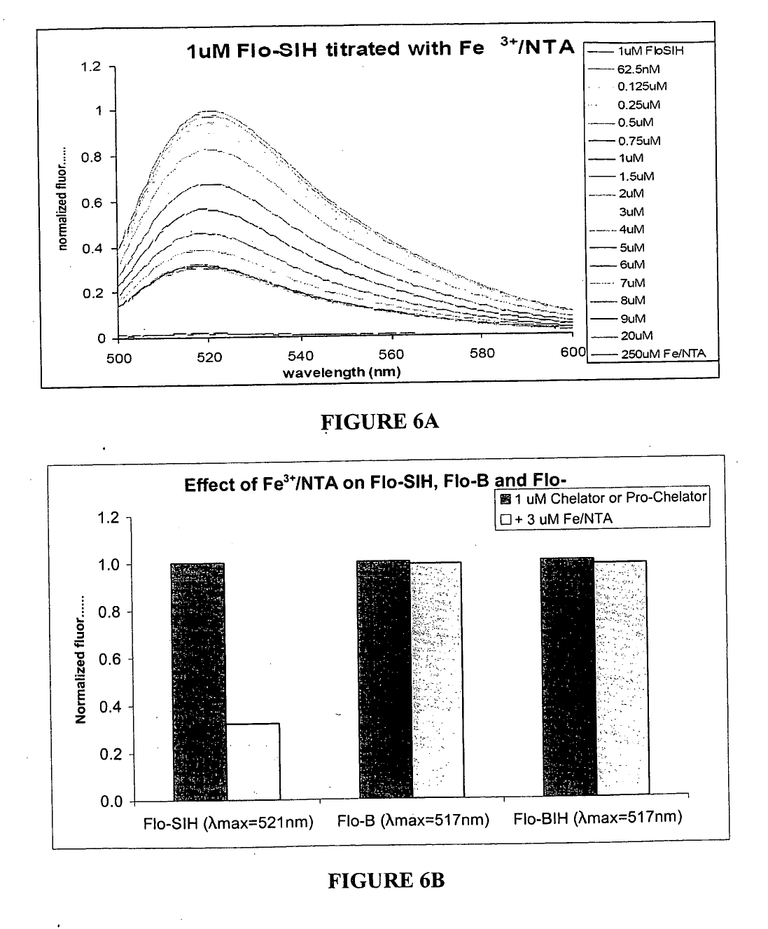

Addition of Fe3+ quenches the fluorescence of Flo-SIH, but induces no change in Flo-B or Flo-BIH. All solutions were prepared in Chelex-treated (i.e. metal free) 10 mM HEPES/lOOmM NaCl buffered at pH 7.00. When excited at 495 nm, all derivatives are highly fluorescent. As iron was added (as an Fe³⁺(NTA) complex to keep iron soluble), Flo-SIH showed significant quenching of its emission at 521 nm (FIG. 6A). However, iron has no effect on the nonchelating control probe, Flo-BIH, or the masked pro-chelator, Flo-B (FIG. 6B).

### Metal-Dependent Ouenching of Flo-SIH

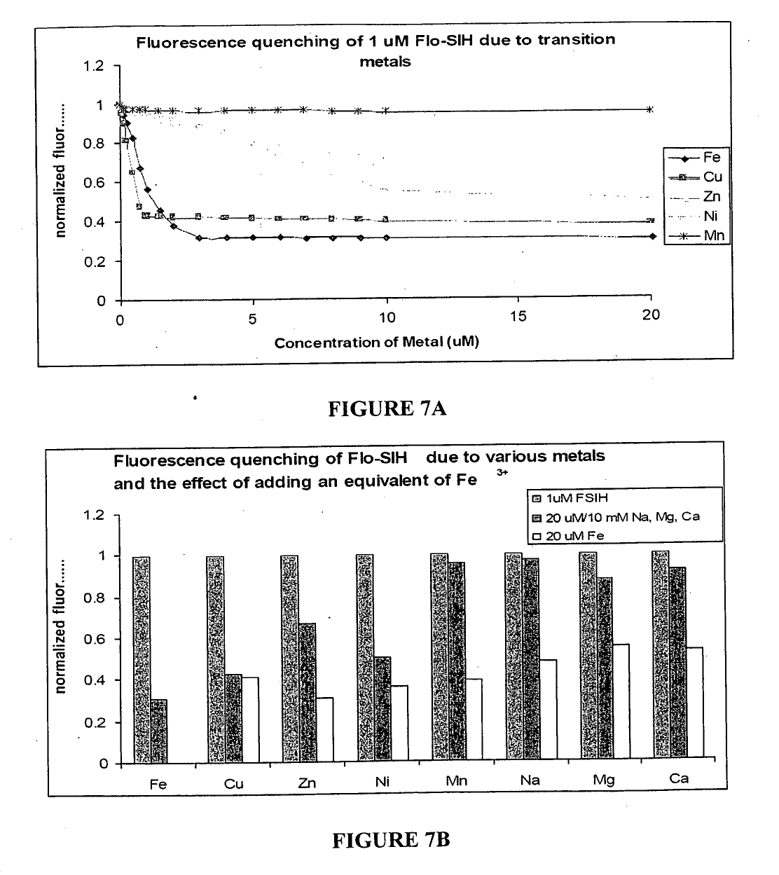

1 uM solutions of Flo-SIH were titrated with a range of metal cations to study their effect on fluorescence quenching (FIG. 7A). All solutions were prepared in 10 mM HEPES/100 mM NaCi buffered at pH 7.00. Cu²⁺, N²⁺, Zn²⁺, and Mn2+ were studied up to concentrations of 20 uM, while Na+, Mg²⁺, and Ca²⁺ were studied up to concentrations of 10 mM. Importantly, the fluorescence of Flo-SIH is non-responsive. even to these elevated concentrations of Na, Ca, and Mg. In addition to Fe³⁺, Cu²⁺ also results in a significant fluorescence quenching response, while Mn²⁺ has no effect. Both Zn²⁺ and Ni²⁺ show some response, but at 5× higher concentration. Upon adding an equal concentration of iron to the solutions, most showed a significant change to the fluorescent signal, indicating that Fe³+competes for binding. The only metal ion among this series that cannot be differentiated from Fe is Cu²⁺.

### REFERENCES

1. Esposito, B. P. et al., A Review of Fluorescence Methods for Assessing Labile Iron in Cells and Biological Fluids. Anal. Biochem. 2002, 304, 1-18.

2. Fan, L.-J.; Wayne E. Jones, J., A Highly Selective and Sensitive Inorganic/Organic Hybrid Polymer Fluorescence “Turn-on” Chemosensory System for Iron Cations. J. Am. Chem. Soc. 2006, 128, (21), 6784-6785.

3. Epsztejn, S. et al., Fluorescence Analysis of the Labile Iron Pool of Mammalian Cells. Anal. Biochem. 1997, 248, 31-40.

4. Summer, J. P.; Kopelman, R., Alexa Fluor 488 as an iron sensing molecule and its application in PEBBLE nanosensors. The Analyst 2005, 130, 528-533.

5. Glickstein, H. et al., Intracellular labile iron pools as direct targets of iron chelators: a fluorescence study of chelator action in living cells. Blood 2005, 106, (9), 3242-3250.

6. Kakhlon, O.; Cabantchik, Z. I., The labile iron pool: Characterization, measurement, and participation in cellular processes. Free Radical Biol. Med. 2002, 33, (8), 1037-1046.

7. Kress, G. J. et al., The relationship between intracellular free iron and cell injury in cultured neurons, astrocytes, and oligodendrocytes. J Neurosci. 2002, 22, (14), 5848-5855.

8. Rauen, U. et al., Assessment of chelatable mitochondrial iron by using mitochondrion-selective fluorescent iron indicators with different iron-binding affinities. Chembiochem 2007, 8, (3), 341-352.

9. Petrat, F. et al., The chelatable iron pool in living cells: A methodically defined quantity. Biol. Chem. 2002, 383, (3-4), 489-502.

10. Ma, Y. M. et al., Chelation and determination of labile iron in primary hepatocytes by pyridinone fluorescent probes. Biochem. J 2006, 395, 49-55.

11. Esposito, B. P. et al., A review of fluorescence methods for assessing labile iron in cells and biological fluids. Anal. Biochem. 2002, 304, (1), 1-18.

12. Hasinoff, B. B., The intracellular iron sensor calcein is catalytically oxidatively degraded by iron(II) in a hydrogen peroxide-dependent reaction. J Inorg. Biochem. 2003, 95, (2-3), 157-164.

13. Komatsu, T. et al., Design and Synthesis of an Enzyme Activity-Based Labeling Molecule with Fluorescence Spectral Change. J Am. Chem. Soc. 2006, 128, (50), 15946-15947.

14. Berthelot, T. et al., Synthesis of N-epsilon-(7-diethylarninocoumarin-3-carboxyl)- and N-epsilon-(7-methoxycoumarin-3-carboxyl)-L-Fmoc lysine as tools for protease cleavage detection by fluorescence. J Pept. Sci. 2005, 11, (3), 153-160.

15. Duggan, P. J.; Tyndall, E. M., Boron acids as protective agents and catalysts in synthesis. J Chem. Soc., Perkin Trans 2002, (1), 1325-1339.

The foregoing is illustrative of the present invention, and is not to be construed as limiting thereof. The invention is defined by the following claims, with equivalents of the claims to be included therein.

That which is claimed is:

1. A fluorescent probe compound of Formula Ia or Formula Ib: 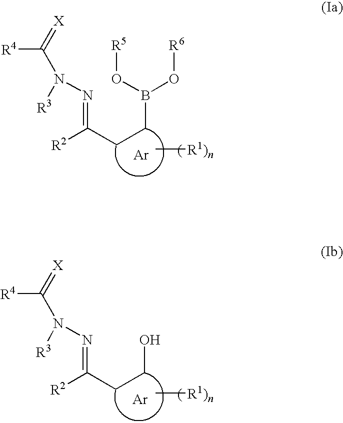
wherein:
R⁴ is A- or A-L-;
X is O or S;
Ar is aryl;
n is an integer from 1 to 4;
each R¹ is independently selected from the group consisting of: alkyl, alkenyl, alkynyl, cycloalkyl, cycloalkylalkyl, cycloalkylalkenyl, cycloalkylalkynyl, heterocyclo, heterocycloalkyl, heterocycloalkenyl, heterocycloalkynyl, aryl, arylalkyl, arylalkenyl, arylalkynyl, heteroaryl, heteroarylalkyl, heteroarylalkenyl, heteroarylalkynyl, alkoxy, halo, mercapto, azido, cyano, formyl, carboxylic acid, hydroxyl, nitro, acyl, aryloxy, alkylthio, amino, alkylamino, arylalkylamino, disubstituted amino, acylamino, acyloxy, ester, amide, sulfoxyl, sulfonyl, sulfonate, sulfonic acid, sulfonamide, urea, alkoxylacylamino, and aminoacyloxy;
R² and R³ are each independently H, alkyl, or hydroxy;
R⁵ and R⁶ are independently selected H, alkyl, or haloalkyl, or together form an alkylene bridge (optionally containing a fused aryl ring), which alkylene bridge may be unsubstituted or substituted from 1 to 4 times with alkyl, halo, a fused aryl ring (such as a fused phenyl), A′-, or A′-L′-,
or each of R⁵ or R⁶ is an independently selected from A′- and A′-L′-;
L and L′ are linking groups; and
A and A′ are first and second fluorophores, which fluorophores are members of a fluorescence resonance energy transfer (FRET) fluorophore pair; or a physiologically-acceptable salt thereof.

2. The compound of claim 1, wherein said compound is a compound of Formula Ia, and R⁵ and R⁶ form an alkylene bridge, which alkylene bridge is substituted from 1-4 times with A′-, or A′-L′-.

3. The compound of claim 1, wherein said compound is a compound of Formula Ia, and both of R⁵ and R⁶ is independently selected from A′- and A′-L′-.

4. The compound of claim 1, wherein said compound is a compound of Formula Ia, and one of R⁵ and R⁶ is independently selected from A′- and A′-L′-.

5. The compound of claim 1, wherein said compound is a compound of Formula Ia, and R⁵ and R⁶ are independently selected H, alkyl, or haloalkyl, or together form an alkylene bridge, which alkylene bridge may be unsubstituted or substituted from 1 to 4 times with alkyl, halo, or a fused aryl ring.

6. The compound of claim 1, wherein said compound is a compound of Formula Ib.

7. The compound of claim 1 having the structure of Formula A, B, C, A′, B′ or C′: 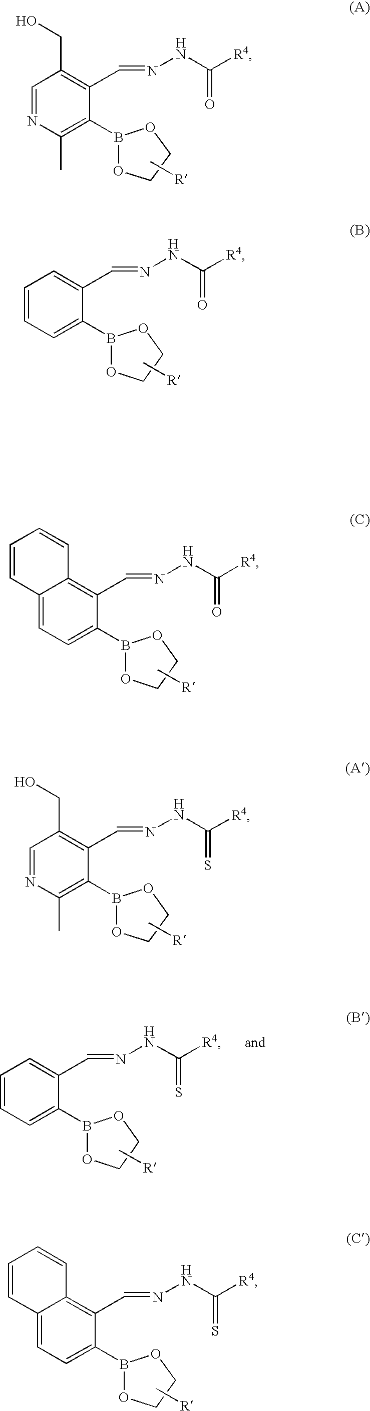
where R⁴ is A- or A-L- as given above, and R′ is A′- or A′-L′- as given above.

8. The compound of claim 1, wherein each member of said FRET pair is independently selected from the group consisting of fluorescin dyes, coumarin dyes, xanthine dyes, rhodamine dyes, and cyanine dyes.

9. The compound of claim 1, wherein each member of said FRET pair is independently selected from the group consisting of sulfonated cyanine dyes, sulfonated rhodamine dyes, and sulfonated carbocyanine dyes.

10. The compound of claim 1, wherein R² is methyl.

11. The compound of claim 1, wherein R³ is methyl.

12. A method of detecting the presence or absence of hydrogen peroxide in a cell or composition, comprising:
administering a compound of any preceding claim to a cell, or adding a compound of any preceding claim to a composition, under conditions in which said compound is cleaved by hydrogen peroxide in said cell or composition;
exciting one member of said FRET pair (A, A′); and
detecting emission from the other member of said FRET pair, wherein emission from said other member of said FRET pair is reduced by the cleavage of said compound by hydrogen peroxide in said cell or composition.

13. A method of detecting the presence or absence of iron or copper in a cell or composition, comprising:
administering a compound of any preceding claim to a cell or composition;
exciting said fluorophore A; and
detecting the presence or absence of emission from said fluorophore A; wherein emission from fluorophore A is quenched by the presence of iron or copper in said cell or composition.

14. The compound of claim 1 conjugated to iron or copper.

15. A method of detecting the presence or absence of an iron or copper chelating agent in a cell or composition, comprising:
administering a compound of claim 14 to a cell or composition, and
detecting fluorescence from said compound, where said fluorescence is quenched in the absence of said chelating agent.

16. A kit comprising a compound of claim 1, and/or instructions for carrying out a method of detecting the presence or absence of hydrogen peroxide, iron or copper therewith, optionally in a common package or container.
